(19) United States
(12) Patent Application Publication
(10) Pub. No.: US 2022/0176059 A1
(43) Pub. Date: Jun. 9, 2022

(54) SYSTEM, METHOD AND APPARATUS FOR DYNAMIC OXYGEN CONSERVER WITH INHALATION SENSOR

(71) Applicant: INCOBA, LLC, Wildwood, MO (US)

(72) Inventor: Alonzo C. AYLSWORTH, Wildwood, MO (US)

(73) Assignee: INCOBA, LLC, Wildwood, MO (US)

(21) Appl. No.: 17/601,543

(22) PCT Filed: Apr. 10, 2020
(86) PCT No.: PCT/US20/27733

§ 371 Date: Oct. 5, 2021

Related U.S. Application Data
(60) Provisional application No. 62/832,695, filed on Apr. 11, 2019.

(51) Int. Cl. A61M 16/06; A61M 16/10; G01L 9/00

(52) U.S. Cl. CPC A61M 16/0677; A61M 2016/0018; G01L 9/0072; A61M 16/101

(57) ABSTRACT

A dynamic oxygen conserver includes a housing and an inhalation sensor coupled to the housing. The sensor has a printed circuit board (PCB) assembly with a first PCB for a breathing side for a user of the dynamic oxygen conserver. A second PCB is coupled to the first PCB. The second PCB is for an atmosphere side opposite the breathing side. In addition, a spacer assembly is coupled between the first and second PCB. The spacer assembly has a metallized diaphragm this is dynamically responsive to breathing by the user of the dynamic oxygen conserver.

### CROSS-REFERENCE TO RELATED APPLICATIONS

This application claims priority to and the benefit of U.S. Prov. Pat. App. No. 62/832,695, filed Apr. 11, 2019, which is incorporated herein by reference in its entirety.

### BACKGROUND

### 1. Field of the Disclosure

This disclosure generally relates to the delivery of oxygen to a user and, in particular, to an improved system, method and apparatus for a dynamic oxygen conserver with an inhalation sensor.

### 2. Description of the Related Art

Research shows that most people have uneven nasal airflow. In particular, many people have a dominant naris and nasal blockage, either structural or from viruses, colds, allergies, etc. According to one study, 80% of the population has a nasal cycle. The nasal cycle is a phenomenon where the nasal passages alternate congestion and decongestion in cycles. It typically occurs all of the time, day and night. A pattern of blockage can occur in a range from about 30 minutes to four hours.

Conventional oxygen devices and nasal cannulas, however, split oxygen delivery approximately 50% to each nostril, which is a cannula style unchanged since the year 1948. That means they can be delivering oxygen to blocked nasal passages, which wastes the dose. To compensate, the oxygen amount may have to be doubled, which is not only wasteful, it's expensive. With nasal blockage occurring most of the time, users are challenged to get their entire dose. Thus, improvement in oxygen devices continue to be of interest.

### SUMMARY

Embodiments of system, method and apparatus for a dynamic oxygen conserver with an inhalation sensor are disclosed. For example, the dynamic oxygen conserver can include a housing and an inhalation sensor coupled to the housing. The sensor can have a printed circuit board (PCB) assembly. The PCB assembly can include a first PCB configured to be a breathing side for a user of the dynamic oxygen conserver. A second PCB can be coupled to the first PCB. The second PCB can be configured to be an atmosphere side opposite the breathing side. In addition, a spacer assembly can be coupled between the first PCB and the second PCB. The spacer assembly can include a metallized diaphragm configured to be dynamically responsive to breathing by the user of the dynamic oxygen conserver.

An alternate embodiment of a dynamic oxygen conserver can include a housing and an inhalation sensor coupled to the housing having a PCB assembly. The PCB assembly can include a base PCB configured to be a breathing side for the user of the dynamic oxygen conserver. First and second PCBs can be coupled opposite the base PCB and configured to be an atmosphere side opposite the breathing side. In addition, a spacer assembly can be coupled between the base PCB and the first and second PCBs. The spacer assembly can include a metallized diaphragm configured to be dynamically responsive to breathing by the user. The base, first and second PCBs can have holes extending therethrough and aligned with portions of the diaphragm. The holes for the base PCB can be configured to be fluidically coupled to respective nostrils and a mouth of the user.

### BRIEF DESCRIPTION OF THE DRAWINGS

For a detailed description of example embodiments, reference will now be made to the accompanying drawings in which:

FIG. 1 is an isometric view of an embodiment of a dynamic oxygen conserver.

FIG. 2 is an isometric view of an embodiment of an inhalation sensor for the dynamic oxygen conserver of FIG. 1.

FIG. 3 is a top view of the sensor of FIG. 2.

FIG. 4 is a side view of the sensor of FIG. 2.

FIG. 5 is a front view of the sensor of FIG. 2.

FIG. 6 is a rear view of the sensor of FIG. 2.

FIG. 7 is a front view of a proximal portion of the sensor of FIG. 2.

FIG. 8 is a sectional side view of the proximal portion of FIG. 7.

FIG. 9 is an exploded isometric view of an embodiment of the sensor of FIG. 2.

FIG. 10 is a front view of an embodiment of a proximal PCB of the sensor of FIG. 2.

FIG. 11 is a rear view of the proximal PCB of FIG. 10.

FIG. 12 is a front view of an embodiment of a distal PCB of the sensor of FIG. 2.

FIG. 13 is a rear view of the distal PCB of FIG. 12.

FIG. 14A is a partially exploded isometric view of layers of an embodiment of a spacer assembly for the sensor of FIG. 9.

FIG. 14B is a reverse view of an assembled version of the spacer assembly of FIG. 14A.

FIGS. 15A-15C are isometric, front and bottom views, respectively, of an alternate embodiment of a dynamic oxygen conserver.

FIG. 16A is a front view of another embodiment of an inhalation sensor.

FIG. 16B is a rear view of the sensor of FIG. 16A.

FIG. 16C is an exploded view of the sensor of FIG. 16A, including spacers and PCBs.

FIG. 16D includes rear views of PCB assemblies of the sensor of FIG. 16A.

FIG. 17A is another embodiment of a PCB assembly.

FIG. 17B is a partially exploded view of the PCB assembly of FIG. 17A.

FIG. 18A is an alternate embodiment of a PCB assembly.

FIG. 18B is a partially exploded view of the PCB assembly of FIG. 18A.

FIG. 19A is a front view of an embodiment of a spacer.

FIG. 19B is a rear view of the spacer of FIG. 19A.

FIG. 19C is an enlarged view of a corner of the spacer of FIG. 19B.

FIG. 19D is an exploded view of the spacer of FIG. 19A.

FIG. 20A is a front view of another embodiment of a spacer.

FIG. 20B is a rear view of the spacer of FIG. 20A.

FIG. 20C is an enlarged view of a corner of the spacer of FIG. 20B.

FIG. 20D is an exploded view of the spacer of FIG. 20A.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Each of the following patent assets is incorporated herein by reference in its entirety.

| Assigned | Country | Patent Name | Filing Date | Issue Date | U.S. Pat. No. |
|---|---|---|---|---|---|
| AirMatrix | US | Method and System for | 28 Mar. 2006 | 11 Sep. 2007 | 7,267,123 |
| | | Measuring Airflow of | | | |
| | | Nares | | | |
| AirMatrix | US | Method and System of | 29 Oct. 2003 | 7 Mar. 2006 | 7,007,692 |
| | | Sensing Airflow and | | | |
| | | Delivering Therapeutic | | | |
| | | Gas to a User | | | |
| AirMatrix | US | Method and System for | 9 Jul. 2003 | 27 Jun. 2006 | 7,066,180 |
| | | Measuring Airflow of | | | |
| | | Nares | | | |
| Acoba | US | Nasal Cannula | 8 Feb. 2005 | 7 Mar. 2006 | 7,007,694 |
| Acoba | US | Hose connection system | 31 May 2007 | 16 Jun. 2015 | 9,056,179 |
| | | for narially sensitive | | | |
| | | diagnostic devices | | | |
| Incoba, | Stated | Method and System of | 7 Oct. 2015 | Pending | PCT/US15/54391 |
| LLC | countries | Sensing Airflow and | | | |
| | worldwide | Delivering Therapeutic | | | |
| | | Gas to a User | | | |

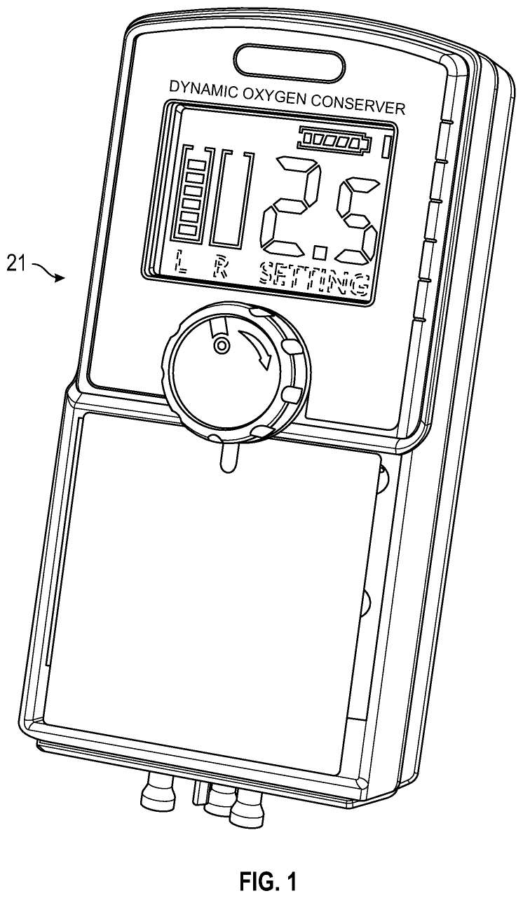

FIG. 1 depicts an embodiment of a dynamic oxygen conserver 21 that can respond to dynamic changes in the breathing (e.g., nasal) resistance to oxygen consumption by a user, as described in the patent assets listed herein. Unlike most static oxygen conservers, the dynamic oxygen conserver 21 can use a cannula system that can detect and selectively deliver oxygen to the naris of the user. Users can get their prescribed dosage without wasting oxygen through delivery to the occluded side. The dynamic oxygen conserver 21 can readily adapt to the nasal cycle described herein. The dynamic oxygen conserver 21 can determine the most patent naris and deliver the oxygen dose instantly, without wasting oxygen sent to the occluded side. The dynamic oxygen conserver 21 can prevent under and over titration, provide consistent, predictable therapy, and decrease waste, thereby greatly extending the range of an oxygen system. See, e.g., www.dynaris.com.

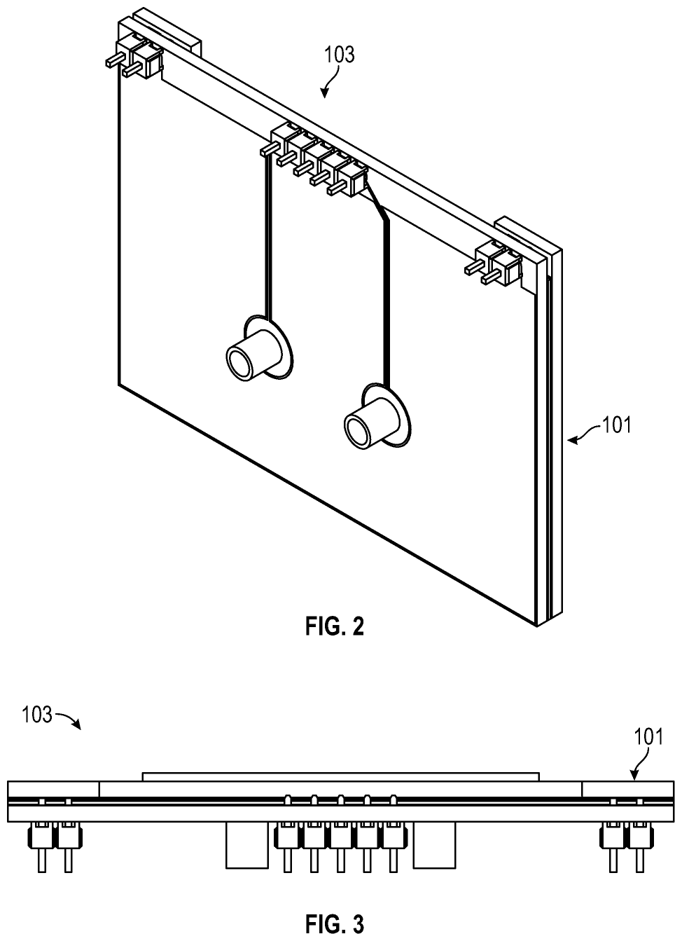

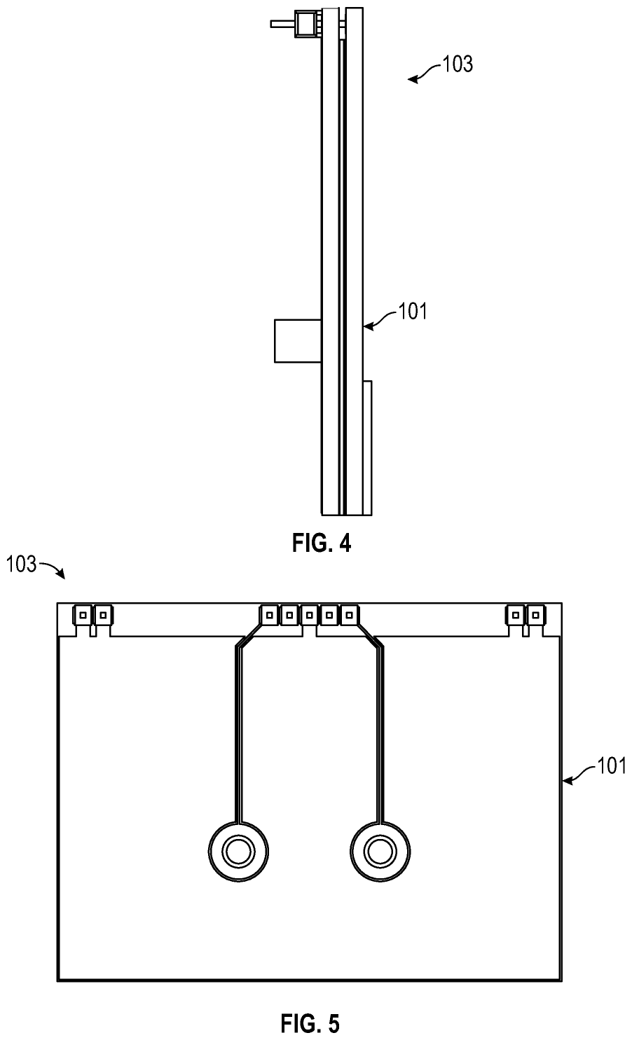

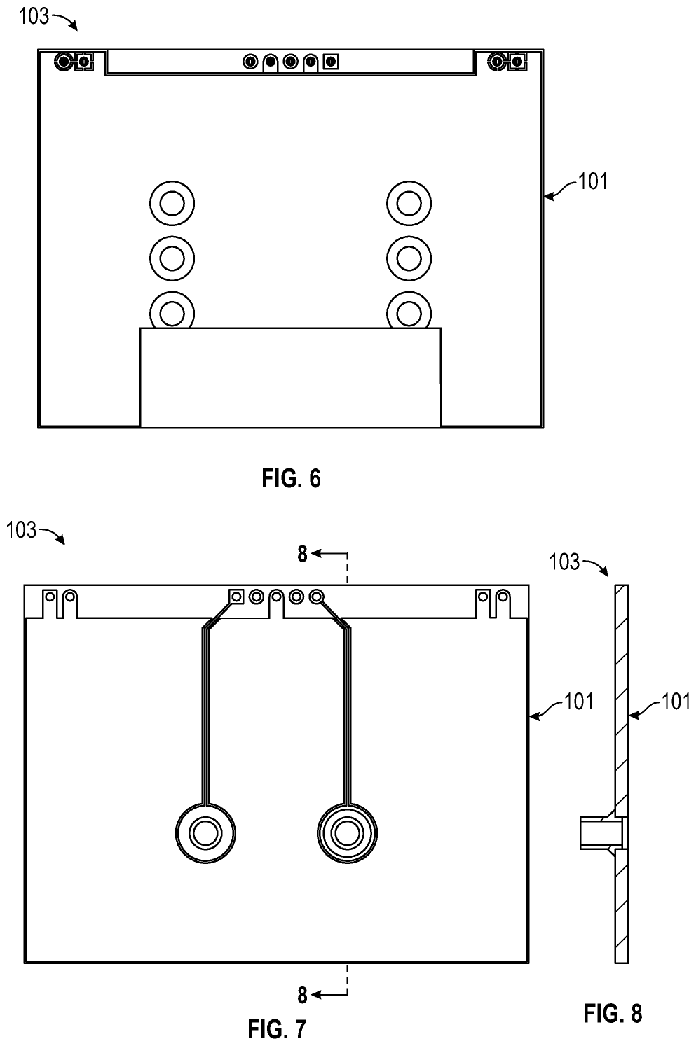

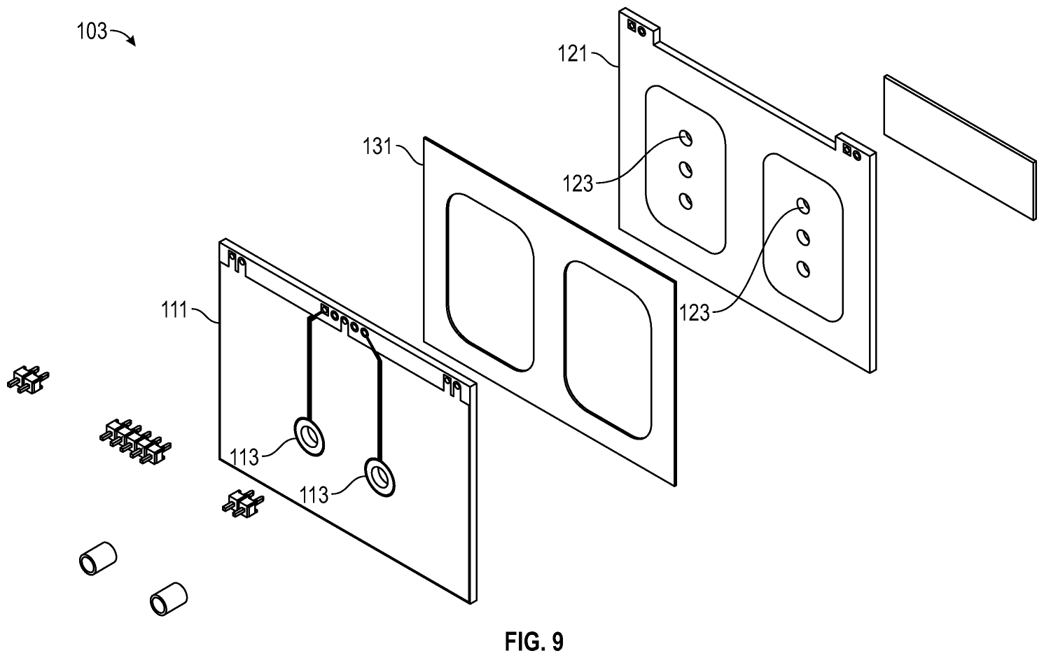

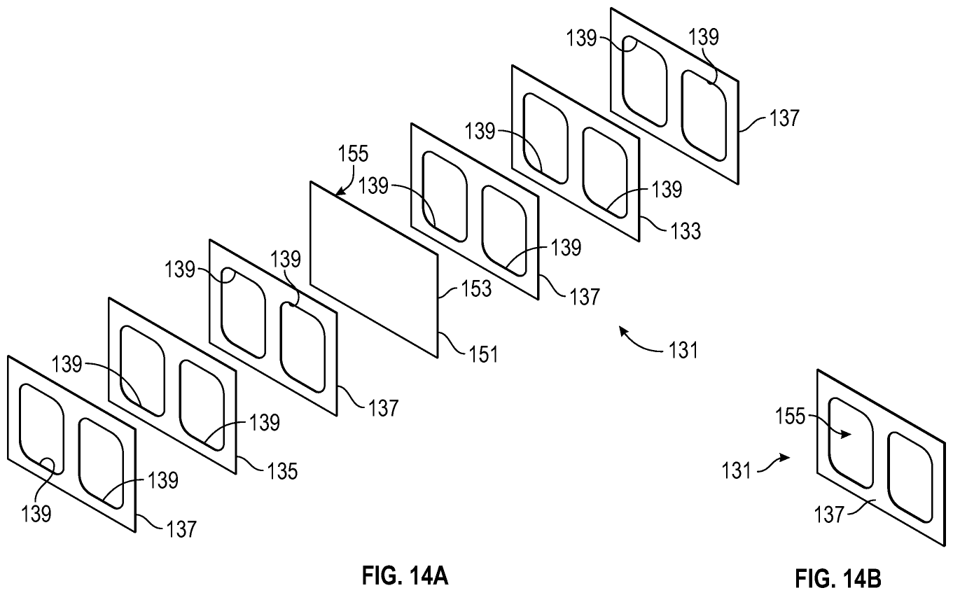

FIGS. 2-14 depict embodiments of an inhalation sensor 101 for the dynamic oxygen conserver 21 (FIG. 1). The inhalation sensor 101 can include a printed circuit board (PCB) assembly 103. The PCB assembly 103 can include a first PCB 111 (FIG. 9) and a second PCB 121 coupled to the first PCB 111. Versions can include a spacer assembly 131 located between the first PCB 111 and the second PCB 121. The spacer assembly 131 can have a diaphragm 151 (FIG. 14), such as a metallized diaphragm, which can be configured to be dynamically responsive to breathing by a user of the dynamic oxygen conserver 21. Embodiments of the PCB assembly 103 can be configured to sense electrical capacitance based on dynamic movement of the diaphragm 151 in response to breathing by a user.

Embodiments of the first PCB 111 can be configured to be proximal to the user. In some examples, the first PCB 111 comprises holes 113 extending therethrough and aligned with portions of the metallized diaphragm 151. A version of the holes 113 can include a pair of holes 113 configured to be fluidically coupled to respective nostrils of the user.

Embodiments of the second PCB 121 can be configured to be distal to the user. The second PCB 121 can include holes 123 extending therethrough and aligned with portions of the metallized diaphragm 151. The holes 123 can include sets of holes 123 configured to communicate with an atmosphere in which the user is located.

In some versions of the inhalation sensor 101 (FIGS. 14A and 14B), the diaphragm 151 can have a base layer 153 having a metallic coating 155. The view in FIG. 14A faces the user side, while the view in FIG. 14B faces the atmosphere side. Examples of the metallic coating 155 can be located on only one side of the base layer 153 and can be configured to be located distal to the user. The base layer 153 can comprise a film, such as a non-metallic film. One example of the base layers 153 can be a polyester film, such as Mylar. Versions of the base layer 153 can have a thickness of about 0.03 mm, or a thickness in a range of about 0.01 mm to about 0.05 mm. In one example, the metallic coating 155 can be vacuum-deposited at a thickness of, for example, about 0.006 mm, or about 0.012 mm, or a thickness in a range of about 0.003 mm to about 0.02 mm. In some versions, the metallic coating 155 can comprise aluminum, silver, or a combination thereof.

Embodiments of the spacer assembly 131 can comprise a laminate having a plurality of layers. For example, the plurality of layers can include spacers 133, 135 and adhesive sheets 137. In some versions, each spacer 133, 135 and adhesive sheet 137 can have holes 139 configured to be aligned with portions of the diaphragm 151. Embodiments of the spacers 133, 135 can have identical or different thicknesses. For example, the spacer 133 (e.g., on the atmosphere side) can have a thickness of about 0.03 mm, or a thickness in a range of about 0.01 mm to about 0.2 mm. Versions of the spacer 135 (e.g., on the breathing side) can have a thickness of about 0.25 mm, or a thickness in a range of about 0.05 mm to about 0.6 mm. The spacers 133 can comprise a film, such as a non-metallic film. In one example, the spacers 133 comprise a polyester film, such as Mylar.

Versions of each of the adhesive sheets 137 can comprise a thickness of about 0.05 mm, or a thickness in a range of about 0.03 mm to about 0.07 mm. In some embodiments, each of the adhesive sheets 137 can comprise adhesive on proximal and/or distal surfaces thereof. Each of the adhesive sheets 137 can include comprises a base layer comprising a film, such as a non-metallic film like polyester film. The adhesive sheets 137 an adhesive, such as a medical grade adhesive (e.g., acrylic). The adhesive sheets 137 can be identical to or different from each other.

The inhalation sensor 101 can have a selected construction. For example, from a proximal side (i.e., the user side, or breathing side) to a distal side (i.e., the atmosphere side, opposite the user), the spacer assembly 131 (FIG. 14A, from left to right) can include a first adhesive sheet 137, spacer 135, a second adhesive sheet 137, the metallized diaphragm 151 (with metallic coating 155 on the atmosphere side, opposite the user), a third adhesive sheet 137, spacer 133, and a fourth adhesive sheet 137. Embodiments of the spacer assembly 131 can have more or fewer components. FIG. 14B illustrates the view of the atmosphere side (opposite the user).

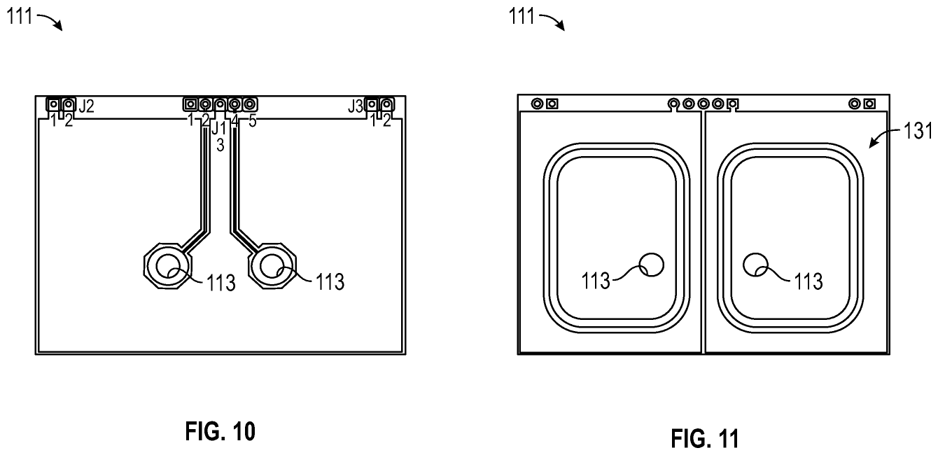

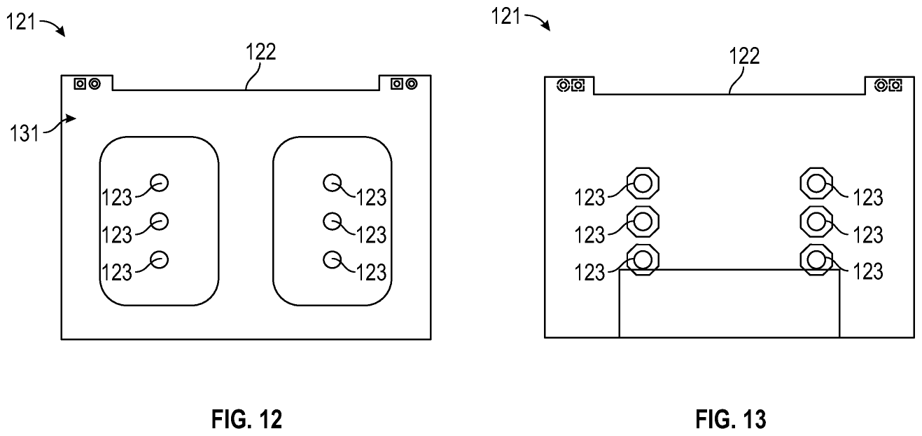

FIGS. 10 and 11 illustrate embodiments having connectors J1, J2 and J3. The connector J1 can have a plurality of pins comprising, for example, pin 1 as a left sensor shield, pin 2 as left sensor electrode, pin 3 as analog ground, pin 4 as right sensor electrode, and pin 5 as right sensor shield. Examples of each of connectors J2 and J3 can include pins 1 and 2, each as analog grounds. In one version, connectors J2 and J3 can pass through both PCB 111 and 121. FIGS. 12 and 13 illustrate an embodiment of PCB 121 having a notch 122 for connector J1.

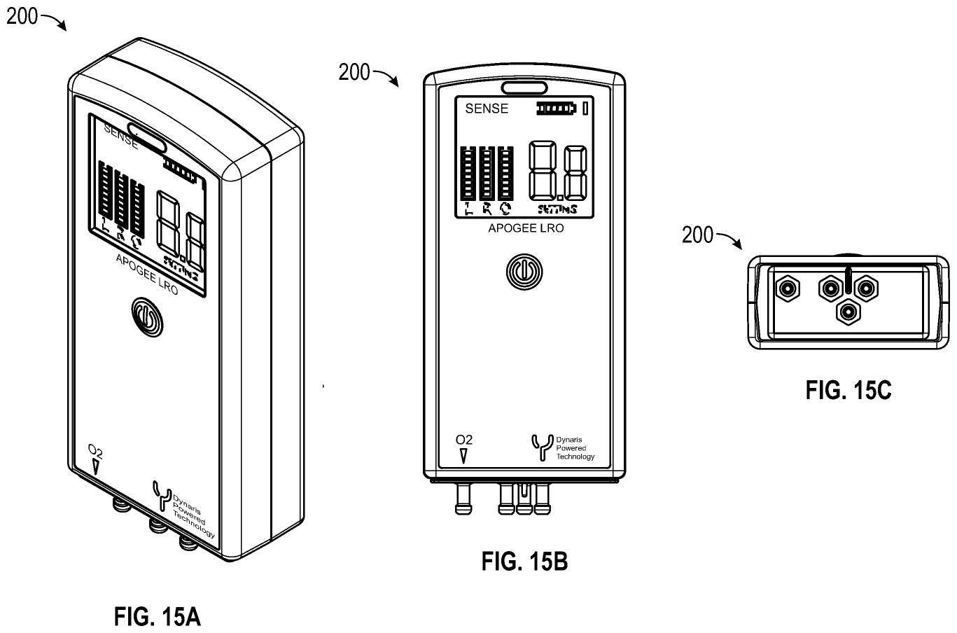

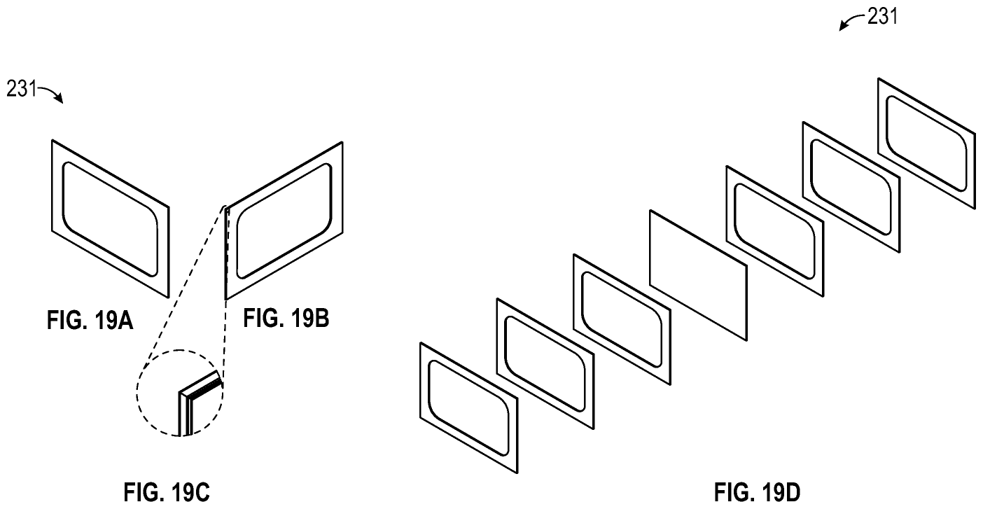

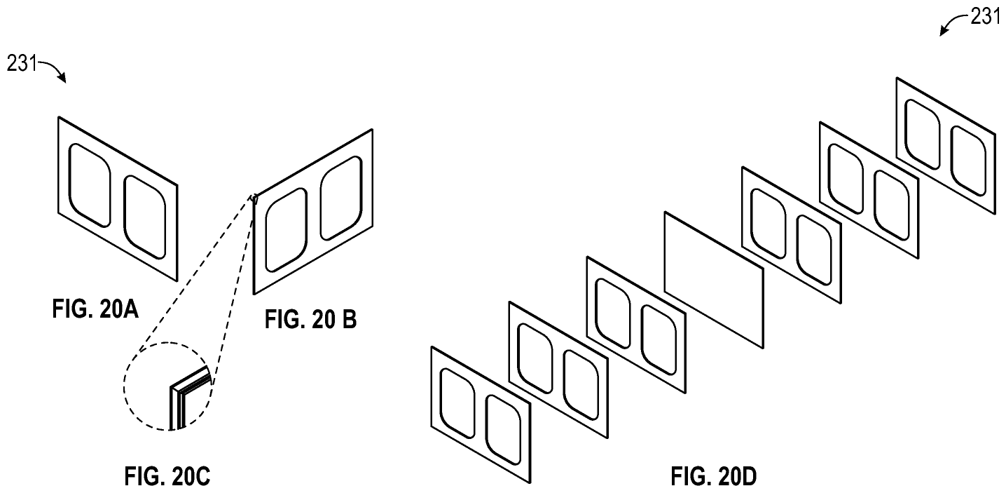

FIGS. 15-20 depict other embodiments of a dynamic oxygen conserver 200. For example, FIG. 15 depicts an embodiment of a dynamic oxygen conserver 200 that can respond to dynamic changes in the nasal and oral breathing resistance to oxygen consumption by a user, as described herein and in the patent assets listed above. Unlike most static oxygen conservers, the dynamic oxygen conserver 200 can use a cannula system that can detect and selectively deliver oxygen to the naris and mouth of the user. Users can get their prescribed dosage without wasting oxygen through delivery to an occluded portion. The dynamic oxygen conserver 200 can readily adapt to the nasal cycle or oral breathing. The dynamic oxygen conserver 200 can determine the most patent naris and deliver the oxygen dose instantly, without wasting oxygen sent to occluded portion(s). The dynamic oxygen conserver 200 can prevent under and over titration, provide consistent, predictable therapy, and decrease waste, thereby greatly extending the range of an oxygen system. See, e.g., www.dynaris.com. Other than being adapted for both nasal and oral breathing, the dynamic oxygen conserver 200 and its components can be identical to or differ from the dynamic oxygen conserver 21.

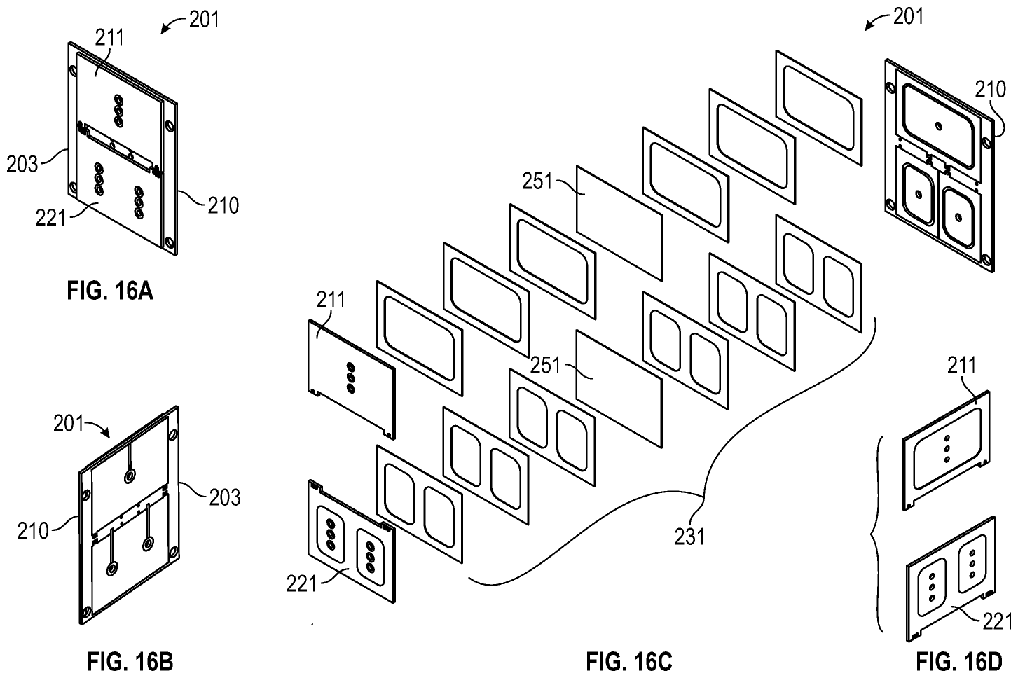

Some embodiments of the dynamic oxygen conserver 200 can include an inhalation sensor 201 (FIG. 16). Versions of the dynamic oxygen conserver 200 can be configured like the dynamic oxygen conserver 21, except for both nasal and oral breathing. Similarly, the inhalation sensor 201 can be configured like the inhalation sensor 103, except for nasal and oral breathing.

Embodiments of the inhalation sensor 201 can include a printed circuit board (PCB) assembly 203 (FIGS. 16A-D). FIGS. 16A, 16C and 16D are views from the atmosphere or non-breathing side. FIG. 16B is a view from the user or breathing side. The PCB assembly 203 can include a base PCB 210 for the user side, and first and second PCBs 211, 221 (FIGS. 16-18) for the atmosphere side. The first and second PCBs 211, 221 can be coupled adjacent to each other, as shown. Versions can include one or more spacer assemblies 231 (FIGS. 16-20) located between the base PCB 210 and the first and second PCBs 211, 221. The spacer assemblies 231 can have a diaphragm 251 (FIG. 16C), such as a metallized diaphragm. The diaphragm 251 can be configured to be dynamically responsive to breathing by a user of the dynamic oxygen conserver 200. Embodiments of the PCB assembly 203 can be configured to sense electrical capacitance based on dynamic movement of the diaphragm 251 in response to nasal and/or oral breathing by a user.

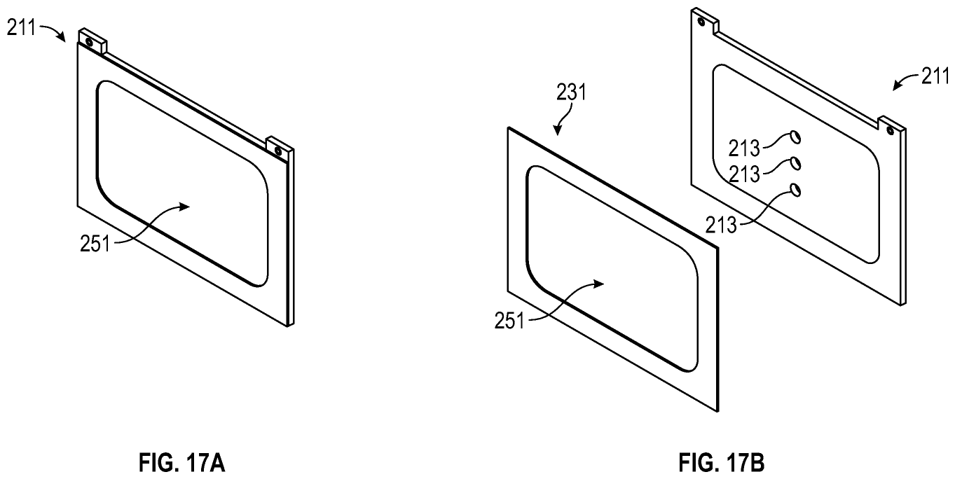

Embodiments of the first PCB 211 (FIG. 17) can be configured to be proximal to the user or designated for a breathing side of the user. In some examples, the first PCB 211 comprises one or more holes 213 extending therethrough and aligned with portions of the metallized diaphragm 251. A version of the holes 213 can include a plurality of holes 213 configured to be fluidically coupled to a mouth of the user.

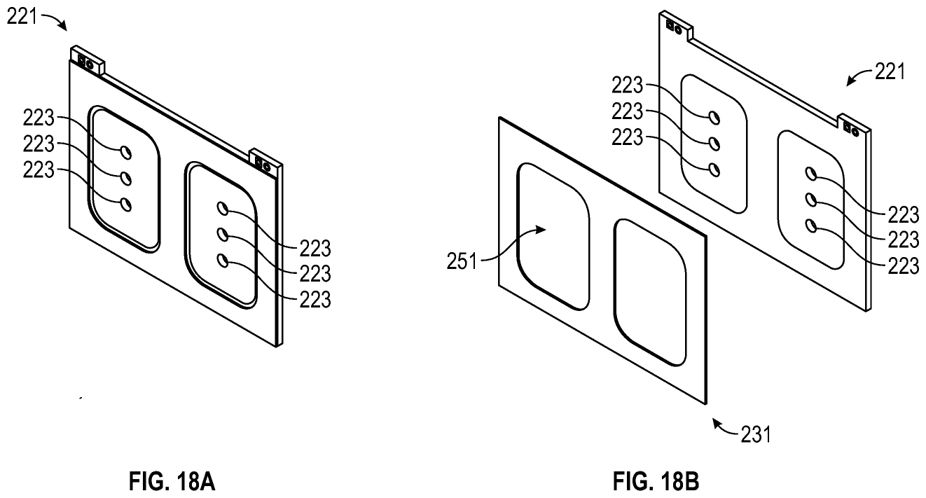

Embodiments of the second PCB 221 (FIG. 18) can be configured to be proximal to the user. The second PCB 221 can include holes 223 extending therethrough and aligned with portions of its own metallized diaphragm 251. The holes 223 can include sets of holes 223. In FIG. 16A, the side of the PCB assembly 201 shown can be configured to communicate with the surrounding atmosphere in which the user is located. In FIG. 16B, the opposite side of the PCB assembly 201 can be configured to be a breathing side for the user.

Some versions of the inhalation sensor 201 can be identical to or differ from the inhalation sensor 101, including parts, materials and construction. The diaphragm 251 can be configured like diaphragm 151, including a base layer having a metallic coating. Examples of the diaphragm 251 can be identical to or different from diaphragm 151 as described herein. Similarly, embodiments of the spacer assembly 231 can be identical to or differ from spacer assembly 131 as described herein.

Other embodiments can include one or more of the following items.

1. A dynamic oxygen conserver, comprising:
A housing;
an inhalation sensor coupled to the housing and comprising a printed circuit board (PCB) assembly, the PCB assembly comprising:
   a first PCB configured to be a breathing side for a user of the dynamic oxygen conserver;
   a second PCB coupled to the first PCB, the second PCB is configured to be an atmosphere side opposite the breathing side;
   a spacer assembly coupled between the first PCB and the second PCB, the spacer assembly comprising a metallized diaphragm configured to be dynamically responsive to breathing by the user of the dynamic oxygen conserver.

2. The inhalation sensor of any of these items, wherein the first PCB is configured to be proximal to the user, the first PCB comprises one or more holes extending therethrough and aligned with portions of the metallized diaphragm, and the one or more holes are configured to be fluidically coupled to respective nostrils and a mouth of the user.

3. The inhalation sensor of any of these items, wherein the second PCB is configured to be distal to the user, the second PCB comprises one or more holes extending therethrough and aligned with portions of the metallized diaphragm, and the one or more holes are configured to communicate with an atmosphere in which the user is located.

4. The inhalation sensor of any of these items, wherein the metallized diaphragm comprises a base layer having a metallic coating.

5. The inhalation sensor of any of these items, wherein the metallic coating is located only on one side of the base layer and is configured to be located opposite of the user.

6. The inhalation sensor of any of these items, wherein the base layer comprises a non-metallic film and has a thickness in a range of about 0.01 mm to about 0.05 mm, and the metallic coating has a metallic coating thickness in a range of about 0.0003 mm to about 0.01 mm.

7. The inhalation sensor of any of these items, wherein the metallic coating comprises at least one of aluminum or silver.

8. The inhalation sensor of any of these items, wherein the spacer assembly comprises a laminate having a plurality of layers.

9. The inhalation sensor of any of these items, wherein the plurality of layers comprises spacers and adhesive sheets.

10. The inhalation sensor of any of these items, wherein each spacer and adhesive sheet comprises holes configured to be aligned with portions of the metal diaphragm.

11. The inhalation sensor of any of these items, wherein the spacers have different thicknesses.

12. The inhalation sensor of any of these items, wherein a first spacer has a first thickness in a range of about 0.01 mm to about 0.2 mm, and a second spacer has a second thickness of about 0.05 mm to about 0.6 mm.

13. The inhalation sensor of any of these items, wherein the spacers comprise non-metallic film.

14. The inhalation sensor of any of these items, wherein each of the adhesive sheets comprises a same thickness in a range of about 0.03 mm to about 0.07 mm.

15. The inhalation sensor of any of these items, wherein each of the adhesive sheets comprises adhesive on proximal and distal surfaces thereof.

16. The inhalation sensor of any of these items, wherein each of the adhesive sheets comprises a base layer comprising non-metallic film and medical grade adhesive.

17. The inhalation sensor of any of these items, wherein from a proximal side to a distal side, the spacer assembly comprises:
a first adhesive sheet;
a first spacer;
a second adhesive sheet;
the metallized diaphragm;
a third adhesive sheet;
a second spacer; and
a fourth adhesive sheet.

18. The inhalation sensor of any of these items, wherein the PCB assembly is configured to sense electrical capacitance based on dynamic movement of the metallized diaphragm.

19. A dynamic oxygen conserver, comprising:
a housing;
an inhalation sensor coupled to the housing, the inhalation sensor comprising a printed circuit board (PCB) assembly, the PCB assembly comprising:
   a base PCB configured to be a breathing side for a user of the dynamic oxygen conserver;
   first and second PCBs coupled opposite the base PCB and configured to be an atmosphere side opposite the breathing side;
   a spacer assembly coupled between the base PCB and the first and second PCBs, the spacer assembly comprising a metallized diaphragm configured to be dynamically responsive to breathing by the user, wherein the base, first and second PCBs comprise holes extending therethrough and aligned with portions of the metallized diaphragm, and the holes for the base PCB are configured to be fluidically coupled to respective nostrils and a mouth of the user.

Many of the electrical connections in the drawings are shown as direct couplings having no intervening devices, but not expressly stated as such in the description above. Nevertheless, this paragraph shall serve as antecedent basis in the claims for referencing any electrical connection as “directly coupled” for electrical connections shown in the drawing with no intervening device(s).

The above discussion is meant to be illustrative of the principles and various embodiments of the present invention. Benefits, other advantages, and solutions to problems have been described above with regard to specific embodiments. However, the benefits, advantages, solutions to problems, and any feature(s) that can cause any benefit, advantage, or solution to occur or become more pronounced are not to be construed as a critical, required, sacrosanct or an essential feature of any or all the claims. Numerous variations and modifications will become apparent to those skilled in the art once the above disclosure is fully appreciated. It is intended that the following claims be interpreted to embrace all such variations and modifications.

What is claimed is:

1. A dynamic oxygen conserver, comprising:
A housing;
an inhalation sensor coupled to the housing and comprising a printed circuit board (PCB) assembly, the PCB assembly comprising:
a first PCB configured to be a breathing side for a user of the dynamic oxygen conserver;
a second PCB coupled to the first PCB, the second PCB is configured to be an atmosphere side opposite the breathing side;
a spacer assembly coupled between the first PCB and the second PCB, the spacer assembly comprising a metallized diaphragm configured to be dynamically responsive to breathing by the user of the dynamic oxygen conserver.

2. The inhalation sensor of claim 1, wherein the first PCB is configured to be proximal to the user, the first PCB comprises one or more holes extending therethrough and aligned with portions of the metallized diaphragm, and the one or more holes are configured to be fluidically coupled to respective nostrils and a mouth of the user.

3. The inhalation sensor of claim 1, wherein the second PCB is configured to be distal to the user, the second PCB comprises one or more holes extending therethrough and aligned with portions of the metallized diaphragm, and the one or more holes are configured to communicate with an atmosphere in which the user is located.

4. The inhalation sensor of claim 1, wherein the metallized diaphragm comprises a base layer having a metallic coating.

5. The inhalation sensor of claim 4, wherein the metallic coating is located only on one side of the base layer and is configured to be located opposite of the user.

6. The inhalation sensor of claim 5, wherein the base layer comprises a non-metallic film and has a thickness in a range of about 0.01 mm to about 0.05 mm, and the metallic coating has a metallic coating thickness in a range of about 0.0003 mm to about 0.01 mm.

7. The inhalation sensor of claim 5, wherein the metallic coating comprises at least one of aluminum or silver.

8. The inhalation sensor of claim 1, wherein the spacer assembly comprises a laminate having a plurality of layers.

9. The inhalation sensor of claim 8, wherein the plurality of layers comprises spacers and adhesive sheets.

10. The inhalation sensor of claim 9, wherein each spacer and adhesive sheet comprises holes configured to be aligned with portions of the metal diaphragm.

11. The inhalation sensor of claim 9, wherein the spacers have different thicknesses.

12. The inhalation sensor of claim 11, wherein a first spacer has a first thickness in a range of about 0.01 mm to about 0.2 mm, and a second spacer has a second thickness of about 0.05 mm to about 0.6 mm.

13. The inhalation sensor of claim 11, wherein the spacers comprise non-metallic film.

14. The inhalation sensor of claim 11, wherein each of the adhesive sheets comprises a same thickness in a range of about 0.03 mm to about 0.07 mm.

15. The inhalation sensor of claim 11, wherein each of the adhesive sheets comprises adhesive on proximal and distal surfaces thereof.

16. The inhalation sensor of claim 11, wherein each of the adhesive sheets comprises a base layer comprising non-metallic film and medical grade adhesive.

17. The inhalation sensor of claim 11, wherein from a proximal side to a distal side, the spacer assembly comprises:
a first adhesive sheet;
a first spacer;
a second adhesive sheet;
the metallized diaphragm;
a third adhesive sheet;
a second spacer; and
a fourth adhesive sheet.

18. The inhalation sensor of claim 1, wherein the PCB assembly is configured to sense electrical capacitance based on dynamic movement of the metallized diaphragm.
